(19) United States
(12) Patent Application Publication
(10) Pub. No.: US 2016/0063208 A1
(43) Pub. Date: Mar. 3, 2016

(54) METHOD AND APPARATUS FOR THE TRICHOTOMOUS IDENTIFICATION OF MORBIDITY, MORTALITY AND SURVIVAL WITHOUT NEW MORBIDITY FROM INTENSIVE CARE

(71) Applicant: Children's National Medical Center, Washington, DC (US)

(72) Inventor: Murray M. Pollack, Washington, DC (US)

(73) Assignee: Children's National Medical Center, Washington, DC (US)

(21) Appl. No.: 14/806,222

(22) Filed: Jul. 22, 2015

Related U.S. Application Data
(60) Provisional application No. 62/099,845, filed on Jan. 5, 2015.
(60) Provisional application No. 62/040,244, filed on Aug. 21, 2014.

(51) Int. Cl. G06F 19/00; A61B 5/00

(52) U.S. Cl. CPC G06F 19/3431; A61B 5/7275; G06F 19/3443; A61B 2503/04; A61B 2505/03

(57) ABSTRACT

A method for determining a patient risk in according with the present application comprises obtaining patient information for a patient admitted to a medical facility, the patient information including patient descriptive information, diagnostic information and physiological information, determining a patient score according to the physiological information, and simultaneously determining, by circuitry, a patient morbidity risk and a patient mortality risk for the patient according the patient descriptive information, the diagnostic information and the patient score. The determining of the patient score according to the physiological information may further include identifying a numerical value for the patient descriptive information, the diagnostic information and the physiological information, and determining the patient score by computing the numerical values.

### CROSS-REFERENCE TO RELATED APPLICATIONS

This application is based upon and claims the benefit of priority from U.S. Application No. 62/040,244, filed Aug. 21, 2014, and U.S. Application No. 62/099,845, filed Jan. 5, 2015, the entire contents of which are incorporated herein by reference.

### BACKGROUND

Mortality adjusted for physiological status and other case mix factors has been a core methodology of adult, pediatric and neonatal intensive care assessments for decades. Users of these methods have been early proponents of standardized mortality ratios for quantitative, unit-based quality assessments for both internal and external benchmarking. Case-mix adjusted survival and death rates are primary outcomes for national databases beyond critical care medicine. For example, the Agency for Healthcare Research and Quality and the Centers for Medicare and Medicaid publish hospital mortality rates for common conditions including acute myocardial infarction, stroke, congestive heart failure, pneumonia, hip fractures, and gastrointestinal hemorrhage.

Numerous studies of the nature and characteristics of Pediatric Intensive Care Units (PICUs) have been conducted, including many by the inventor of this application. Such studies routinely emphasize that there is dramatic variability among PICUs in their patient populations, characteristics of care, and even organizational characteristics. Importantly, there is also substantial variability in crude and adjusted mortality and morbidity rates among PICUs.

Mortality rates in pediatric intensive care units have decreased since the original methods adjusting for physiological dysfunction were developed, and medical therapies are increasingly focused on reducing morbidity in survivors. Therapeutic initiatives such as hypothermia, prevention of secondary injury following head trauma, rapid resuscitation of shock, and early thrombolysis therapy are aimed at reducing survivors' morbidity as well as improving survival rates. However, most quantitative outcome assessment methods continue to focus on the dichotomous outcome of survival versus death.

There is a need to address morbidity following pediatric intensive care, especially morbidity that affects functional status. Moreover, given the decreasing PICU mortality rates, the ability to more finely assess outcomes among surviving children in terms of morbidity allows the opportunity to distinguish between different care practices at a more refined level, thereby furthering the opportunity to improve patient outcomes.

Critical Care in general and Pediatric Critical Care in particular have developed excellent measures of severity of illness calibrated to mortality. Mortality prediction models are highly dependent on physiologic status that includes cardiovascular, neurological, respiratory, renal, metabolic, and hematological dysfunction. However, severity may be reflected in morbidity as well as in survival. For example, physiologic dysfunctions such as cardiovascular compromise may cause neurological injury and/or be associated with long-term cardiac compromise. Low blood sugar has a risk for neurological injury. Coagulopathies are associated with intracranial hemorrhage. Morbidity is in the progression of injury resulting from physiologic dysfunction such that morbidity is an intermediate outcome and death is the final outcome. Incorporation of morbidity into severity of illness models encourages uses such as assessment of quality of care, clinical uses such as decision-support aids, and future studies including forecasting economic and social impacts of intensive care based on contemporary patient data.

Recently, a reliable and rapid quantitative assessment of functional status for children analogous to the activities of daily living scale used in adults was developed. This development is especially pertinent to large outcome studies. Previous methods for children were either (a) time consuming to conduct, (b) limited to older infants and children, or simply required too much subjective assessment and future projection by raters (Pediatric Cerebral and Overall Performance Scales). Since the median age of patients in PICUs is less than 3 years in most PICUs, measures that do not include the complete age spectrum are not relevant even to a majority of children. This development of a rapid and reliable method of measuring functional status enables the development and use of methods to assess morbidity risk.

### SUMMARY

New morbidity is associated with physiological status in the cardiovascular, respiratory, neurological and hematological systems in a similar fashion as to that of mortality with physiological status. Morbidity increases as physiological dysfunction increases and then decreases as the physiological dysfunction becomes sufficiently large to change potential morbidities into mortalities.

New morbidity significantly affecting functional status is often an event along the path toward mortality as both outcomes are strongly associated with the degree of physiological alterations. In pediatric critical care, new morbidity assessed by change in functional status is almost twice as common as mortality and could serve as a new, clinically relevant and important outcome for clinical trials and quality studies to supplement the relatively low rate of mortality.

The inventors have determined that morbidity, like mortality, is associated with physiological dysfunction and could be predicted simultaneously with mortality. Assessments of care including quality assessments adjusted for physiological status should include the development of new morbidities as well as mortalities.

The inventors assessed morbidity with the Functional Status Scale (FSS), and defined at an increase of ≧3 from pre-illness to hospital discharge. Physiological status was measured by the Pediatric Risk of Mortality (PRISM) III score.

The inventors developed a multivariate trichotomous prediction model to assess the potential for simultaneous prediction of morbidity and mortality.

In accordance with the present disclosure, the Functional Status Scale, a rapid and reliable measure of functional status can be validated against the Adaptive Behavior Assessment System II as an outcome measure suitable for multi-state outcome assessments of infants and children.

In accordance with the present disclosure, trichotomous outcomes of intensive care (death, survival with new morbidity defined by a significant decrease in functional status, and survival without a new decrement in functional status) can be predicted from acute physiologic status, acute and chronic diagnoses, chronic health status including functional status, and other information derived from the first hours of pediatric intensive care.

In accordance with the present disclosure, there is a significant correlation between hospital discharge functional status and 6-month and 12-month post-discharge functional status for children discharged from the Pediatric ICU.

Accordingly, an objective of the present disclosure is to validate a rapid, and reliable measure of functional status applicable to the full age group of pediatric patients while demonstrating both the feasibility and an excellent likelihood of success.

The objective of the present disclosure is to validate a predictor of three outcome states from pediatric intensive care: death, survival with new morbidity defined by a significant decrease in functional status, and survival without a new decrement in functional status

Another objective of the present disclosure is to classify proportions of patients that fall into the appropriate categories. Many of these children will present to the Pediatric ICU with chronic functional disabilities. Therefore, the predictor must be able to account for both those with existing disabilities on admission and those with new disabilities associated with the current ICU illness. Pediatrics in particular, but also adult medicine would be altered by this conceptual change in anticipated prediction relevance of a severity measure.

Secondary benefits include the provision of a standard physiologic and descriptive database that is designed to provide appropriate baseline data for long-term follow-up studies including long-term functional status and economic issues.

In accordance with the present disclosure, new morbidities associated with physiological status can be modeled simultaneously with mortality utilizing trichotomous outcome models. New morbidities significantly affecting functional status at hospital discharge can be modeled simultaneously with mortality according to factors including physiological status measured by the PRISM III score, age, admission source, and other diagnostic factors. Trichotomous modeling uncovers the phasic association of morbidity risk with physiological status and produces a well-performing model for simultaneous prediction of both morbidity and mortality suitable for risk adjustment in research, quality and other studies.

According to some embodiments, a method for determining a patient risk comprises obtaining patient information for a patient admitted to a medical facility, the patient information including patient descriptive information, diagnostic information and physiological information, determining a patient score according to the physiological information, and simultaneously determining, by circuitry, a patient morbidity risk and a patient mortality risk for the patient according the patient descriptive information, the diagnostic information and the patient score.

The determining of the patient score according to the physiological information may further comprise identifying a numerical value for the patient descriptive information, the diagnostic information and the physiological information, and determining the patient score by computing the numerical values.

The physiological information may include information about a condition of the patient prior to admission to the medical facility. The physiological information may include information about a condition of the patient upon admission to the medical facility. The patient descriptive information may include patient history information.

According to some embodiments, the patient is admitted to an intensive care unit of the medical facility when the patient has a congenital or acquired heart defect, the patient is less than three months of age, and the patient score is determined within a period of 2 hours prior to admission and 4 hours after a cardiac interventional procedure. Further, the information consisting of the new morbidity and mortality risks of the patient may be used by the medical staff/parents/family to better understand the patient's prognosis and to aid in medical decision making.

According to some embodiments, a method for evaluating a quality of care in a hospital comprises collecting patient information of a group of patients treated in the hospital over a period of time, the patient information for each patient of the group of patients including patient descriptive information, diagnostic information, and physiological information, determining, by circuitry, an expected number of patients with new morbidities of the group of patients according to the patient information, determining an actual number of patients with new morbidities of the group of patients according to the patient information, determining, by the circuitry, an expected number of mortalities of the group of patients according to the patient information, determining an actual number of mortalities of the group of patients according to the patient information, and determining, by the circuitry, the quality of care in the hospital according to the expected and actual number of patients with new morbidities and the expected and actual number of mortalities.

According to some embodiments, the physiological information may include information about a condition of the patient prior to admission to the medical facility. The determining of the expected number of patients with new morbidities comprises determining, for each patient of the group of patients, a patient score according to the physiological information, for each patient, simultaneously determining, by the circuitry, a patient morbidity risk and a patient mortality risk of the patient according the patient descriptive information, the diagnostic information and the patient score, and calculating, by the circuitry, the expected number of patients with new morbidities according to the patient morbidity risk of each patient in the group of patients.

According to some embodiments, the determining of the patient score according to the physiological information comprises identifying a numerical value for the patient descriptive information, the diagnostic information and the physiological information, and determining the patient score by computing the numerical values. The physiological information may include information about a condition of the patient upon admission to the medical facility. The patient descriptive information may include patient history information.

An apparatus in accordance with embodiments of the present disclosure comprises circuitry configured to obtain patient information for a patient, the patient information including patient descriptive information, diagnostic information, and physiological information, determine a patient score according to the physiological information, and simultaneously determine a patient morbidity risk and a patient mortality risk for the patient according the patient descriptive information, the diagnostic information and the patient score.

To determine the patient score, the circuitry may be configured to identify a numerical value for the patient descriptive information, the diagnostic information and the physiological information, and determine the patient score by computing the numerical values. The circuitry may be configured to collect patient information for a group of patients over a period of time, the group of patients including the patient, determine an expected number of patients with new morbidities of the group of patients according to the descriptive information, the diagnostic information, and the physiological information, determine an actual number of patients with new morbidities of the group of patients according to the patient descriptive information, the diagnostic information and the physiological information, determine an expected number of mortalities of the group of patients according to the descriptive information, the diagnostic information, and the physiological information, determine an actual number of mortalities of the group of patients according to the patient descriptive information, the diagnostic information and the physiological information, and determine the quality of care in the hospital according to the expected and actual number of patients with new morbidities and the expected and actual number of mortalities.

According to some embodiments, to determine the expected number of patients with new morbidities, the circuitry is further configured to determine, for each patient of the group of patients, a patient score according to the physiological information, simultaneously determine, for each patient, a patient morbidity risk and a patient mortality risk of the patient according the descriptive information, the diagnostic information, and the patient score, and calculate the expected number of patients with new morbidities according to the patient morbidity risk and the patient mortality risk of each patient in the group of patients. When the patient is not admitted to an intensive care unit of the medical facility and the patient is over 3 months of age, the circuitry calculates the patient score within a period of 2 hours prior to admission and 4 hours after admission to the medical facility.

Although this discussion focuses on modeling morbidity and mortality in pediatric ICUs, the modeling also applies to patients in neonatal and adult intensive care units, i.e. such patients have a similar relationship between morbidity and physiological dysfunction.

### BRIEF DESCRIPTION OF THE DRAWINGS

A more complete appreciation of the disclosure and many of the attendant advantages thereof will be readily obtained as the same becomes better understood by reference to the following detailed description when considered in connection with the accompanying drawings, wherein:

FIG. 1 illustrates a flow chart of a method for the determination of a patient simultaneous morbidity and mortality risk.

FIG. 2 illustrates a flow chart of a method for the assessment of a quality of care of a hospital.

FIG. 3 illustrates a flow chart of a method for the allocation of resources in a hospital system.

FIG. 4 illustrates a schematic diagram of an exemplary device in accordance with the present disclosure.

FIG. 5 illustrates a schematic diagram of a data processing system for performing the methods illustrated in FIGS. 1-3.

FIG. 6 illustrates a schematic diagram of CPU 530.

FIG. 7 illustrates a schematic diagram of a plurality of devices connected over a network for performing the methods illustrated in FIGS. 1-3.

### DETAILED DESCRIPTION OF THE PREFERRED EMBODIMENTS

Conceptually, severity of illness may be considered a continuous variable with extremes of outcomes (survival, death) occurring at low and high values. Although the threshold value that determines the outcome may vary from patient to patient, the concept of severity of illness is useful in the treatment of patients in pediatric, neonatal and adult intensive care, utilizing scoring systems such as PRISM, SNAP, APACHE, and many more.

The utilization of current methods for determining a severity of illness have successfully adjusted mortality rates for the severity differences of different populations. However, intermediate outcomes associated with physiologic status (e.g. compromised functional status) may occur between the extremes and at different points on a severity of illness scale.

Physiology-based mortality predictors, such as PRISM, combine a multitude of patient-related variables (e.g. cardiovascular, neurological, metabolic, respiratory, renal, and hematological) in multivariate models to reflect their contribution to mortality risk. Extreme dysfunction of the magnitude that contributes to mortality risk is also associated with potential disability as well as death. For example, neurological injury as well as permanent effects on each individual organ system of a patient may result from these physiologic deviations. Reductions in blood flow evidenced by blood pressure and heart rate changes, poor oxygenation, low blood sugar, poor coagulation, etc., may increase the risk of neurological injury. Acute respiratory, cardiovascular, and renal failures risk long-term organ system dysfunction. Further, physiologic dysfunction is also correlated with length of Pediatric ICU stay, increasing the “opportunities” for morbidities secondary to new insults such as nosocomial infections.

However, until the present work of the inventors, there has been no research into how an initial severity of illness, assessed by routine ICU methods of physiologic status, is related to hospital discharge, intermediate and long-term functional status. The present disclosure addresses this issue by applying severity of illness methodologies to the identification and prediction of hospital discharge functional status which is correlated to intermediate and long-term functional status of patients treated in ICUs.

Measuring functional status and outcomes requires an understanding of the type of functioning that is being assessed. Traditional intelligence scales by themselves would miss many children with severe, non-cognitive dysfunction. Adaptive behavior is a conceptually appealing way to make traditionally adult concepts of disability and dependency relevant in infants and children.

Age-appropriate scales measuring adaptive abilities in daily living, communication, socialization and motor skills are available that span appropriate age groups. The Vineland Adaptive Behavior Scales (VABS), the Adaptive Behavior Scale (ABS) II and the Adaptive Behavior Assessment System (ABAS) II are standard pediatric instruments for classifying individuals into functional categories for outcome evaluation.

The present discussion utilizes the Functional Status Scale (FSS) for determining age appropriate functional status and new decrements in functional status. The VABS score was used for determining criterion validity and the ABAS II was used for validation of the FSS. Other scales may be utilized for the determination of functional status and outcomes of infants, children and adults, such as Glasgow Outcome Scale (GOS), Functional Independence Measure (FIM and weeFIM), the Level of Cognitive Functioning Scale (Los Ranchos Los Amigos Scale), Rappaport's Disability Rating Scale the Pediatric Overall Performance Category (POCP) scale, and the Pediatric Cerebral Performance Category (PCPC) scale.

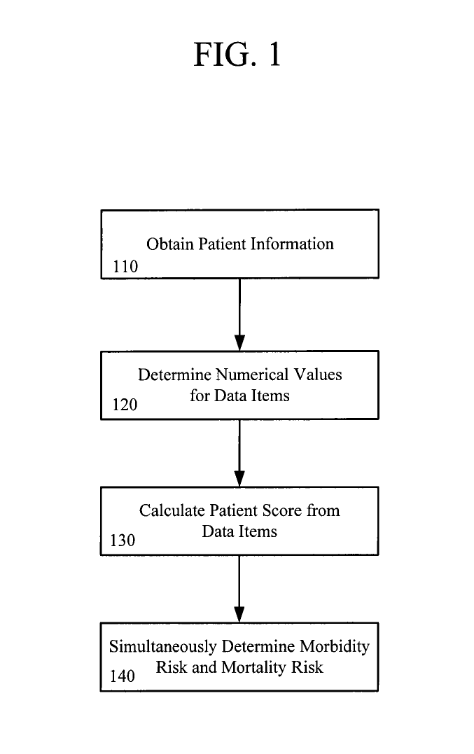

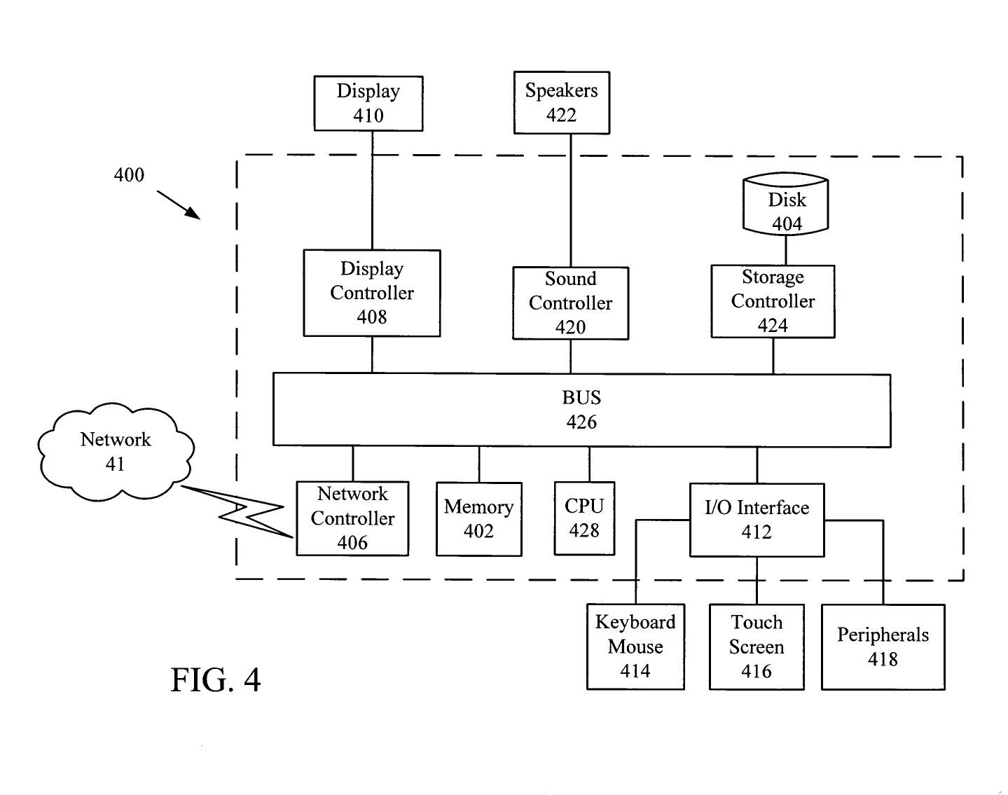

FIG. 1 illustrates a flow chart of a method for the determination of a patient morbidity risk. In an exemplary implementation, method 100 of FIG. 1 is performed by a processor or circuitry of a computer terminal, mobile apparatus, or network device. The processor or circuitry will be described later with respect to FIGS. 4-7.

Method 100 begins at step 110, wherein patient information is obtained. In an exemplary implementation, patient information is collected by a medical professional at a time of patient admission, although the patient information may be collected by other means, such as by a parent, relative, doctor, or other professional. Further the patient information may be collected prior to or after patient admission to a hospital or other medical facility.

The patient information may include any or all of patient descriptive information, diagnostic information and physiological information.

Patient descriptive information may include patient history information. Examples of patient descriptive information include general ICU admission data such as: admission date and time; date of birth; elective/emergency status; operative status; clinical service of primary responsibility; previous ICU admissions during the current hospitalization; prior diagnoses; age; catastrophic conditions prior to admission (e.g. respiratory, cardiac arrests); clinical service(s) of primary responsibility; known mental retardation or developmental delay and how determined; the FSS (to be discussed later) prior to ICU admission (from medical record, parents or care provider). Data pertinent to “lead time bias” may include the referring location, and the duration of hospitalization prior to ICU admission (if from the ER, duration of time in the ER).

Diagnostic information of a patient may include any of the following: principle and underlying diagnoses; worst and best neurological functioning (best motor response, GCS, and level of consciousness); iatrogenic states influencing physiologic variables (e.g. pharmacologically induced paralysis and coma); catastrophic events; operative status; and changes in diagnoses, and physiological system(s) of dysfunction requiring admission to the hospital or intensive care unit.

Physiologic information may include measurements such as systolic and diastolic blood pressure, heart rate, mental status, pupillary reflexes, temperature, pH, total CO₂, PCO₂, PaO₂, glucose, potassium, BUN, creatinine, WBC count, platelet count, and prothrombin and partial thromboplastin times (PT/PTT).

Upon collection, the patient information may be entered into a medical database. After the patient information is obtained, the method proceeds to step 120.

In step 120, numerical values are assigned to the physiological information. Numerical values may also be assigned to the patient descriptive information and the diagnostic information.

After numerical values are assigned to the patient information, a patient score is determined in step 130.

In step 130, the patient score is determined. In an exemplary implementation, the patient score is calculated from the numerical values of the physiological information. The patient score may also be calculated from other data items, such as the numerical values of the patient descriptive information and the diagnostic information.

The patient score may be determined by calculating a PRISM III score. For example, the PRISM III score is calculated according to the following (1)-(5).

(1) If the patient is NOT admitted to the intensive care unit for cardiac surgery or an interventional cardiac catheterization, or is over 3 months of age, use period from 2 hours prior to admission to 4 hours after admission to assess laboratory variables and the time period from admission to 4 hours after admission to assess physiological variables.

(2) If the patient is admitted to the intensive care unit for cardiac surgery or an interventional cardiac catheterization and is under 3 months of age, use the following table to determine the time interval to assess the PRISM III score:

TABLE 1

| | ICU length of stay | |
|---|---|---|
| | prior to Cardiac | PRISM III Collection |
| Age at Admission | Intervention | Time Interval |
| <24 hours | ≦12 hours | Admission |
| | 12 hours-10 days | Post-Intervention |
| 24 hours to 10 days | 0-10 days | Post-Intervention |
| | >10 days | Admission |
| 11 days to 30 days | ≦48 hours | Post-Intervention |
| | >48 hours | Admission |
| 31 days to 90 days | ≦48 hours | Post-Intervention if |
| | | cardiac surgery |
| | | Admission if cardiac |
| | | catheterization |
| | >48 hours | Admission |
| >90 days | All | Admission |

The admission time interval refers the period of the 2 hours prior to admission to 4 hours after admission for laboratory data and the first 4 hours of PICU care for other physiological variables. The post-intervention time interval refers to the first 4 hours of PICU care after a cardiac intervention (surgery or interventional catheterization, but not diagnostic catheterization).

(3). The Pediatric Risk of Admission (PRISM) III Score is composed of the following Table 2:

Cardiovascular and Neurologic Vital Signs

| | | |
|---|---|---|
| Systolic | Score = 3 | Score = 7 |
| blood | | |
| pressure | | |
| (mm Hg) | | |
| Neonate | 40-55 | <40 |
| Infant | 45-65 | <45 |
| Child | 55-75 | <55 |
| Adolescent | 65-85 | <65 |
| Temperature | Score = 3 | |
| | <33° C. or >40° C. | |
| Mental | Score = 5 | |
| Status | Stupor/coma or GCS < 8 | |
| Heart rate | Score = 3 | Score = 4 |
| (beats per | | |
| minute) | | |
| Neonate | 215-225 | >225 |
| Infant | 215-225 | >225 |
| Child | 185-205 | >205 |
| Adolescent | 145-155 | >155 |
| Pupillary | Score = 7 | Score = 11 |
| reflexes | One fixed | Both fixed |

Acid-Base, Blood Gases

| | | |
|---|---|---|
| Acidosis | Score = 2 | Score = 6 |
| (pH or | | |
| total CO2) | | |
| pH | 7.0-7.28 | <7.0 |
| CO2 | 5-16.9 | <5 |
| PCO2 | Score = 1 | Score = 3 |
| (mm Hg) | 50-75 | >75 |
| Alkalosis: | Score = 4 | |
| Total | | |
| CO2 | >34 | |
| (mmol/L) | | |
| PaO2 | Score = 3 | Score = 6 |
| (mm Hg) | 42-49 | <42 |

Chemistry Tests

| | |
|---|---|
| Glucose | Score = 2 |
| | >200 mg/dL or >11 mmol/L |
| Potassium | Score = 3 |
| (mmol/L) | >6.9 |
| Blood urea | Score = 3 |
| nitrogen | |
| (BUN) | |
| Neonate | >11.9 mg/dL or >4.3 mmol/L |
| All other | >14.9 mg/dL or >4.3 mmol/L |
| ages | |
| Creatinine | Score = 2 |
| Neonate | >0.85 mg/dL or >75 mmol/L |
| Infant | >0.90 mg/dL or >80 mmol/L |
| Child | >0.90 mg/dL or >80 mmol/L |
| Adolescent | >1.30 mg/dL or >115 mmol/L |

Hematology Tests

| | | | |
|---|---|---|---|
| White blood | Score = 4 | | |
| cell count | <3,000 | | |
| (cells/mm3) | | | |
| Platelet | Score = 2 | Score = 4 | Score = 5 |
| Count (×103 | 100-200 | 50-99 | <50 |
| cells/mm3) | | | |
| Prothrombin | Score = 3 | | |
| time (PR) | | | |
| or Partial | | | |
| thrombo- | | | |
| plastin | | | |
| (time) (PTT) | | | |
| Neonate | PT > 22 or PTT > 85 | | |
| All other | PT > 22 or PTT > 57 | | |
| ages | | | |

GCS = Glasgow Coma Scale.

(4) A neonate is <30 days of age, an infant is 30 days to <12 months of age, a child is 12 months of age to <=144 months of age, and an adolescent is >144 months of age.

(5) The neurological PRISM III is composed of the mental status and pupillary reflex scores and the non-neurological PRISM III is composed of all other PRISM III components.

After calculation of the patient score, the method proceeds to step 140.

In step 140, a morbidity risk and a mortality risk are simultaneously determined. In particular, the morbidity risk and the mortality risk are simultaneously determined according to the patient descriptive information, the diagnostic information and the patient score calculated in step 130.

In an exemplary implementation, morbidity risk increases with increasing PRISM III scores and then decreases as potential morbidities increasingly become mortalities. In the trichotomous relationship assessed in the present disclosure, the relationship of PRISM III with mortality changes very little after accounting for simultaneous morbidity risk. However, the relationship of morbidity with PRISM III changes substantially after accounting for mortality risk; morbidity risk initially increases with higher PRISM III scores, but then decreases with further increases in the PRISM III scores whose mortality risk is high.

The following equation is used for the simultaneously determine a morbidity risk and a mortality risk, as in step 140:

$$P = \frac{1}{1 + {e}^{- ( {β}_{0} + x \cdot β )}}$$

where β₀ is the intercept, β is the coefficient for each of the independent variables, and x is the coefficient. The computation of morbidity risk and mortality risk separately utilizes the morbidity intercept β₀ and the sum of for each independent variable β multiplied by its respective coefficient (x). The value of the categorical variable is assigned 1 if it is present or 0 if the categorical variable is absent. The values of the PRISM III Neurological Score and the PRISM III non-Neurological Score are recorded as the PRISM III score determined by Table 2. Table 3 includes values that demonstrate the statistical precision and reliability of the trichotomous model according to the present disclosure.

TABLE 3
Trichotomous Outcome Model for Simultaneous Prediction of Morbidity and Mortality.

| | | Odds Ratios: | | Odds Ratios: |
|---|---|---|---|---|
| | Morbidity | New Morbidity vs. | Mortality | Death vs. No |
| | Coefficients | No New Morbidity | Coefficients | New Morbidity |
| Predictors | (SE) | (95% CI) | (SE) | (95% CI) |
| Intercept | −3.92 (0.17) | NA | −5.51 (0.27) | NA |
| Age at PICU Admission | | | | |
| 0 day to <14 days | 0.80 (0.23) | 2.23 (1.43, 3.49) | 1.64 (0.27) | 5.14 (3.00, 8.79) |
| 14 days to <1 month | 0.47 (0.44) | 1.61 (0.68, 3.79) | 1.26 (0.56) | 3.53 (1.19, 10.50) |
| 1 month to <12 months | 0.39 (0.14) | 1.48 (1.13, 1.93) | 0.42 (0.21) | 1.52 (1.02, 2.28) |
| >12 months | Reference | Reference | Reference | Reference |
| Admission Source | | | | |
| Direct admission: Referral | 0.76 (0.15) | 2.15 (1.59, 2.90) | 1.09 (0.24) | 2.96 (1.87, 4.70) |
| Hospital | | | | |
| Inpatient Unit: Same Hospital | 0.87 (0.18) | 2.38 (1.67, 3.39) | 1.70 (0.25) | 5.46 (3.33, 8.95) |
| Emergency Department: Same | 0.11 (0.16) | 1.12 (0.81, 1.53) | 0.64 (0.25) | 1.90 (1.16, 3.14) |
| Hospital | | | | |
| OR/PACU for Postoperative | Reference | Reference | Reference | Reference |
| Care | | | | |
| Cardiac Arrest (1) | 0.97 (0.33) | 2.63 (1.38, 5.00) | 1.52 (0.33) | 4.56 (2.40, 8.66) |
| Acute (non-Primary) or Chronic | 0.25 (0.28) | 1.28 (0.74, 2.21) | 0.89 (0.30) | 2.44 (1.36, 4.40) |
| Diagnosis of Cancer (1) | | | | |
| Trauma (1) | 1.18 (0.19) | 3.26 (2.23, 4.77) | 0.81 (0.35) | 2.26 (1.13, 4.51) |
| Primary System of Dysfunction | | | | |
| Cardiovascular/Respiratory | Reference | Reference | Reference | Reference |
| Cancer | 0.73 (0.28) | 2.07 (1.20, 3.59) | 0.90 (0.43) | 2.47 (1.06, 5.74) |
| Low Risk (DKA, Hematologic, | −0.93 (0.31) | 0.39 (0.21, 0.72) | −1.69 (0.61) | 0.18 (0.06, 0.61) |
| Musculoskeletal, Renal) | | | | |
| Neurologic | 0.38 (0.15) | 1.46 (1.08, 1.98) | −0.07 (0.25) | 0.93 (0.57, 1.54) |
| Other | −0.21 (0.23) | 0.81 (0.52, 1.28) | 0.11 (0.31) | 1.11 (0.61, 2.03) |
| Baseline FSS Score | −0.23 (0.13) | 0.80 (0.61, 1.03) | −0.66 (0.19) | 0.52 (0.36, 0.74) |
| Categorized as Good (1, 2) | | | | |
| PRISM III Neurological Score | 0.11 (0.02) | 1.12 (1.08, 1.16) | 0.21 (0.02) | 1.24 (1.19, 1.29) |
| (3, 4) | | | | |
| PRISM in Non-Neurological | 0.09 (0.01) | 1.09 (1.07, 1.12) | 0.18 (0.01) | 1.19 (1.16, 1.23) |
| Score (4) | | | | |

NA = not applicable
(1). Reference is absence of the factor.
(2). Baseline FSS score = 6 or 7.
(3). PRISM III neurological components are pupillary reactions and mental status.
(4). For each one point change.

After the morbidity risk and the mortality risk are determined in step 140, the morbidity risk and mortality risk may be utilized in many ways. For example, the morbidity risk and mortality risk may be utilized for the allocation of hospital resources, such as the allocation of hospital beds in a pediatric intensive care unit. Description of the allocation of beds according to a calculated length of stay of a patient is described in U.S. Pat. No. 5,809,477, issued Sep. 15, 1998, the entirety of which is incorporated by reference.

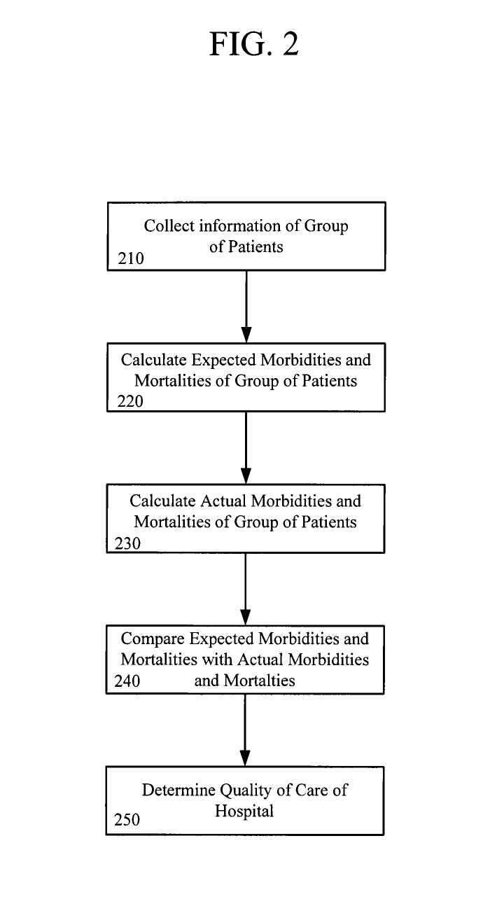

The morbidity risk and mortality risk may be utilized may be also utilized for the determination of a quality of care of a medical facility, as discussed with reference to FIG. 2.

FIG. 2 illustrates a flow chart of a method for the assessment of a quality of care of a hospital. In an exemplary implementation, method 200 of FIG. 2 is performed by a processor or circuitry of a computer terminal, mobile apparatus, or network device. The processor or circuitry will be described later with respect to FIGS. 4-7.

Method 200 begins in step 210, where patient information for a group of patients is collected. For example, step 210 may correspond to step 110 of FIG. 1. In some embodiments, a user may determine an evaluation period for the assessment of the quality of care. The evaluation period may be determined before, concurrently or after step 210. Further, the user may determine a particular group patients in the hospital for which the quality of care may be assessed. For example, only a specific subset of patients from the hospital, such as patients treated within a specific department or by a single doctor, may be selected. Or, the user may determine a single patient for whom the knowledge or morbidity and/or mortality risk may be useful information to the healthcare workers, the patient, and/or the family.

In step 220, an expected number of new morbidities and mortalities in the group of patients is calculated. The calculation of the expected number of new morbidities and mortalities may utilize the simultaneous determination of morbidity and mortality risk as in step 140 of FIG. 1.

In an exemplary implementation, the calculation in step 220 may include the calculation of an expected morbidity rate and an expected mortality rate. From such calculated rates, the expected new morbidities and mortalities may be determined.

The expected morbidity rate of the group of patients may be calculated according to the morbidity risk assessed for each patient of the group of patients. For example, the expected morbidity rate of the group of patients may be calculated according to a summation of the individual patient risks, average, weighted average, median or other statistical calculation of the morbidity risk assessed for each patient of the group of patients.

In step 230, the actual number of new morbidities and mortalities in the group of patients is calculated. In an exemplary implementation, the actual number of new morbidities and mortalities may be determined and the rates calculated using the size of the group. Alternatively, the number of new morbidities and mortalities may be calculated by calculating an actual morbidity rate and an actual mortality rate for the group of patients. From such actual morbidity and mortality rates, the actual number of new morbidities and mortalities may be determined according to a size of the group of patients.

The actual morbidity rate may represent a value equal to a number of patients discharged with impaired functional status divided by a total number of patients discharged by the hospital. The actual morbidity rate of the group of patients may be calculated a predetermined period of time after the entire group of patients has completed treatment or been discharged from the hospital. The actual morbidity rate may be calculated according to a count, percentage, fraction, average, weighted average, median or other statistical calculation of the group of patients. In an exemplary implementation, the actual morbidity rate may be calculated in step 230 according to a same method as the calculation of the expected morbidities as in step 220.

The analysis of actual morbidity status may also include analysis of the relationship of functional status on hospital discharge to initial hospital functional status and occurrences that may influence it. Potential influences on functional status include such items as diagnosis, surgery, physiological instability (PRISM III score), steroid use, sedation, paralysis, length of stay, known complications, etc.

In step 240, the expected number of new morbidities and mortalities and the actual number of new morbidities and mortalities are compared. From such a comparison, the method proceeds to step 250.

In step 250, a quality of care of a hospital is assessed. In an exemplary implementation, a quality of care of the hospital may be determined according to the comparison performed in step 240. Further, the quality of care of the hospital may be determined according to a first ratio between the actual number of new morbidities and mortalities, as calculated in step 230, and the expected number of new morbidities and mortalities, as calculated in step 220.

The actual number of new morbidities divided by the expected number of new morbidities is termed the Standardized Morbidity Ratio. The actual number of mortalities divided by the expected number of mortalities is termed the Standardized Mortality Ratio Ratios significantly greater than 1 indicate less than expected quality of care and ratios significantly less than one indicate better than expected quality of care. The Standardized Morbidity Ratio and the Standardized Mortality Ratio may undergo statistical analysis, most commonly with parametric tests such as the z-score.

Thus, one application of the present disclosure is to improve care. In particular, as a result of the system determining quality of care of a pediatric ICU by way of mortality and morbidity scores for a predetermined time period and comparing these mortality and morbidity scores against a predetermined threshold, it is possible to identify an underperforming pediatric ICU. Once the underperforming ICU is identified, steps can be taken to address these issues. The assessment of under-performance is individualized but may consist of specific care giver performance, policies and procedures, care practices, levels of training, education issues, etc. The system can also be used to identify the same factors that correspond to lower mortality and morbidity scores.

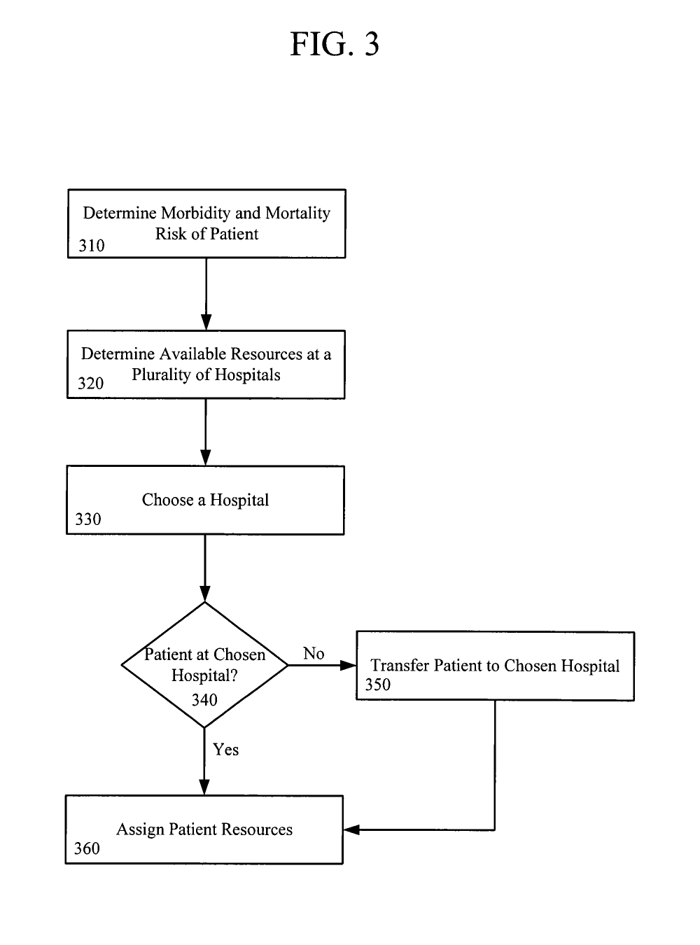

FIG. 3 illustrates another method for the utilization of the morbidity and mortality risks as determined in step 140 of FIG. 1.

In particular, FIG. 3 illustrates a flow chart of a method for the allocation of resources in a hospital system. In an exemplary implementation, method 300 of FIG. 3 is performed by a processor or circuitry of a computer terminal, mobile apparatus, or network device. The processor or circuitry will be described later with respect to FIGS. 4-7.

In step 310, a morbidity risk of a patient is determined. The determination of the morbidity risk of the patient may be performed as in method 100 of FIG. 1.

In step 320, available resources of a plurality of hospitals are determined. For example, in a hospital network that includes three hospitals, the available resources of the three hospitals is determined. In another embodiment, the available resources of a plurality of units or departments within a hospital or a plurality of hospitals are determined. For example, in a hospital network that includes two hospitals, and the hospitals include a total of nine units/departments, the available resources of the two hospitals and the nine units/departments are determined.

In step 330, a particular hospital of the plurality of hospitals is chosen for a newly-admitted patient according to the available resources of the plurality of hospitals. The particular hospital may be chosen according to the patient's morbidity risk, the patient's preferences and/or preferences of the patient's family. Further, a particular unit/department of a hospital may be chosen according to factors such as the patient's morbidity risk, the patient's preferences and/or preferences of the patient's family.

In step 340, it is determined whether the chosen hospital is the hospital at which the patient has been newly-admitted. If it is determined that the chosen hospital is the hospital at which the patient has been newly-admitted, the method proceeds to step 360. If, however, it is determined that the chosen hospital is a different hospital than the one at which the patient has been newly-admitted, the method proceeds to step 350.

In step 350, the newly-admitted patient is relocated to the chosen hospital. After relocation of the patient to the chosen hospital, the method proceeds to step 360.

In step 360, the newly-admitted patient is assigned the hospital resource at the chosen hospital. For example, should the available resource at issue be a bed, the newly-admitted patient is assigned a bed.

In an exemplary implementation, the available hospital resources may be a number of available beds for patients. However, the available hospital resources could be medical equipment (e.g. scanning equipment, life-assist devices such as ventilators and/or blood filters), medical personnel and/or medical supplies.

For example, an available number of hospital beds may be determined according to a difference between a total number of hospital beds and an occupied number of hospital beds. In an exemplary implementation, the available hospital resources are determined according to a request time and date. In such an implementation, the occupied number of hospital beds may be equal to a sum of the number of patients in which the LOS overlaps with the requested time and date.

In another example, an available number of beds in an ICU at a future requested time and date may be determined according to a difference between a total number of hospital beds in the ICU and an expected number of hospital beds to be occupied in the ICU. The expected number of hospital beds to be occupied in the ICU may be equal to a sum of the number of patients in which the LOS overlaps with the requested time and date.

Moreover, in some embodiments, it is determined in step 340 whether or not the particular unit/department at which the patent is newly-admitted is the chosen unit/department. Should the chosen hospital be another unit/department within a same hospital, the patient may be relocated to the other unit/department in step 350 to be then assigned the hospital resource in step 360.

FIG. 4 illustrates device 400 in accordance with the present disclosure. In an exemplary embodiment as illustrated in FIG. 4, device 400 includes a CPU 428 which may perform the methods described above with respect to FIGS. 1-3. Process data and instructions for the calculations and other method steps as described above with respect to FIGS. 1-3 may be stored in memory 402. These processes and instructions may also be stored on a storage medium disk 404 such as a hard drive (HDD) or portable storage medium or may be stored remotely. Moreover, the instructions may be stored on CDs, DVDs, in FLASH memory, RAM, ROM, PROM, EPROM, EEPROM, hard disk or any other information processing device with which device 400 communicates, such as a server (remote or local) or computer.

Further, the process data and instructions may be provided as a utility application, background daemon, or component of an operating system, or combination thereof, executing in conjunction with CPU 428 and an operating system such as Microsoft Windows 7, UNIX, Solaris, LINUX, Apple MAC-OS and other systems known to those skilled in the art.

The hardware elements in order to achieve device 400 may be realized by various circuits or circuitry elements. For example, CPU 428 may be a Xenon or Core processor from Intel of America or an Opteron processor from AMD of America, or may include other types of processors or circuits recognized by one of ordinary skill in the art. Alternatively, the CPU 428 may be implemented on an FPGA, ASIC, PLD or using discrete logic circuits, as one of ordinary skill in the art would recognize. Further, CPU 428 may be implemented as multiple processors cooperatively working in parallel to perform the instructions of the inventive processes described above.

Device 400 in FIG. 4 may include a network controller 406 for interfacing with network 41. As can be appreciated, the network 41 can be a public network, such as the Internet, or a private network such as an LAN or WAN network, or any combination thereof and can also include PSTN or ISDN sub-networks. The network 41 can also be wired, such as an Ethernet network, or can be wireless such as a cellular network including EDGE, 3G and 4G wireless cellular systems. Network 41 can also be encompassed via WiFi, Bluetooth, or any other wireless form of communication.

Device 400 may further include a display controller 408 for interfacing with display 410, such as an LCD monitor. A general purpose I/O interface 412 interfaces with a keyboard and/or mouse 414 as well as a touch screen panel 416 on or separate from display 410. General purpose I/O interface also connects to a variety of peripherals 418 including printers and scanners. A sound controller 420 may be provided in device 400, to interface with speakers/microphone 422 to provide sound output or alerts.

The general purpose storage controller 424 connects the storage medium disk 404 with communication bus 426, which may be an ISA, EISA, VESA, PCI, or similar, for interconnecting all of the components of the device 400. A description of the general features and functionality of the display 410, keyboard and/or mouse 414, as well as the display controller 408, storage controller 424, network controller 406, sound controller 420, and general purpose I/O interface 462 is omitted herein for brevity as these features are known.

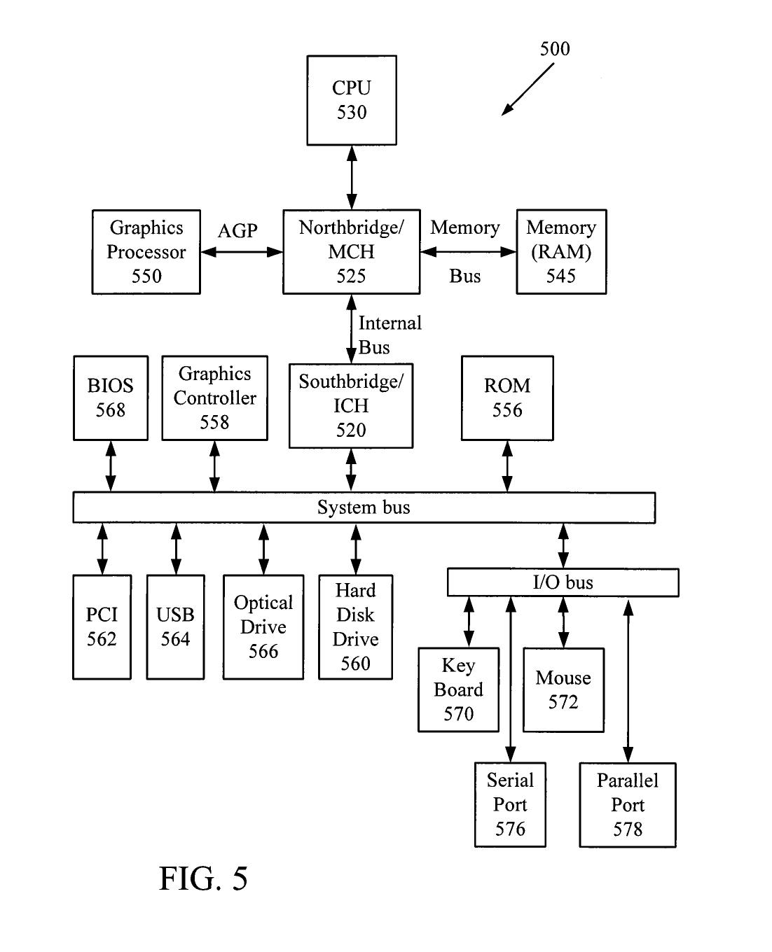

The exemplary circuit elements described in the context of the present disclosure may be replaced with other elements and structured differently than the examples provided herein. Moreover, circuitry configured to perform features described herein may be implemented in multiple circuit units (e.g., chips), or the features may be combined in circuitry on a single chipset, as shown in FIG. 5.

FIG. 5 illustrates a schematic diagram of a data processing system, according to certain embodiments, for performing the methods described above with respect to FIGS. 1-3. The data processing system is an example of a computer in which code or instructions implementing the processes of the illustrative embodiments may be located.

In FIG. 5, data processing system 500 employs a hub architecture including a north bridge and memory controller hub (NB/MCH) 525 and a south bridge and input/output (I/O) controller hub (SB/ICH) 520. The central processing unit (CPU) 530 is connected to NB/MCH 525. The NB/MCH 525 also connects to the memory 545 via a memory bus, and connects to the graphics processor 550 via an accelerated graphics port (AGP). The NB/MCH 525 also connects to the SB/ICH 520 via an internal bus (e.g., a unified media interface or a direct media interface). The CPU Processing unit 530 may contain one or more processors and even may be implemented using one or more heterogeneous processor systems.

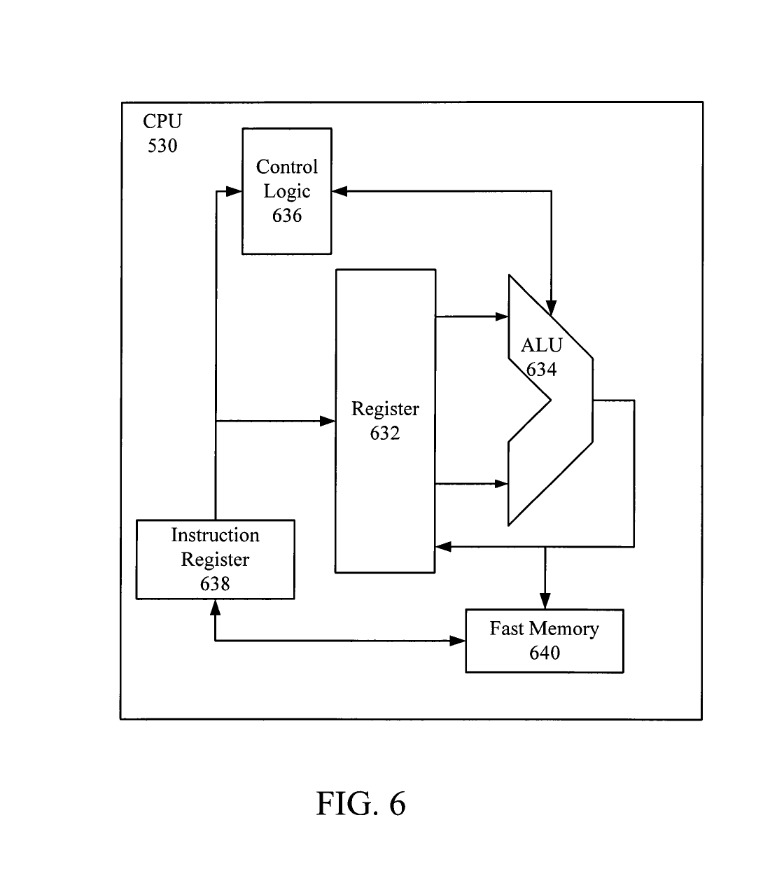

For example, FIG. 6 shows one implementation of CPU 530. In one implementation, the instruction register 638 retrieves instructions from the fast memory 640. At least part of these instructions are fetched from the instruction register 638 by the control logic 636 and interpreted according to the instruction set architecture of the CPU 530. Part of the instructions can also be directed to the register 532. In one implementation the instructions are decoded according to a hardwired method, and in another implementation the instructions are decoded according to a microprogram that translates instructions into sets of CPU configuration signals that are applied sequentially over multiple clock pulses. After fetching and decoding the instructions, the instructions are executed using the arithmetic logic unit (ALU) 634 that loads values from the register 632 and performs logical and mathematical operations on the loaded values according to the instructions. The results from these operations can be feedback into the register and/or stored in the fast memory 640. According to certain implementations, the instruction set architecture of the CPU 530 can use a reduced instruction set architecture, a complex instruction set architecture, a vector processor architecture, a very large instruction word architecture. Furthermore, the CPU 530 can be based on the Von Neuman model or the Harvard model. The CPU 530 can be a digital signal processor, an FPGA, an ASIC, a PLA, a PLD, or a CPLD. Further, the CPU 530 can be an x86 processor by Intel or by AMD; an ARM processor, a Power architecture processor by, e.g., IBM or any other CPU architecture.

Referring again to FIG. 5, the data processing system 500 can include that the SB/ICH 520 is coupled through a system bus to an I/O Bus, a read only memory (ROM) 556, universal serial bus (USB) port 564, a flash binary input/output system (BIOS) 568, and a graphics controller 558. PCI/PCIe devices can also be coupled to SB/ICH 520 through a PCI bus 562.

The PCI devices may include, for example, Ethernet adapters, add-in cards, and PC cards for notebook computers. The Hard disk drive 560 and CD-ROM 566 can use, for example, an integrated drive electronics (IDE) or serial advanced technology attachment (SATA) interface. In one implementation the I/O bus can include a super I/O (SIO) device.

Further, the hard disk drive (HDD) 560 and optical drive 566 can also be coupled to the SB/ICH 520 through a system bus. In one implementation, a keyboard 570, a mouse 572, a parallel port 578, and a serial port 576 can be connected to the system bust through the I/O bus. Other peripherals and devices that can be connected to the SB/ICH 520 using a mass storage controller such as SATA or PATA, an Ethernet port, an ISA bus, a LPC bridge, SMBus, a DMA controller, and an Audio Codec.

Moreover, the present disclosure is not limited to the specific circuit elements described herein, nor is the present disclosure limited to the specific sizing and classification of these elements. For example, the skilled artisan will appreciate that the circuitry described herein may be adapted based on changes to circuit configuration, power source sizing and/location and network connection.

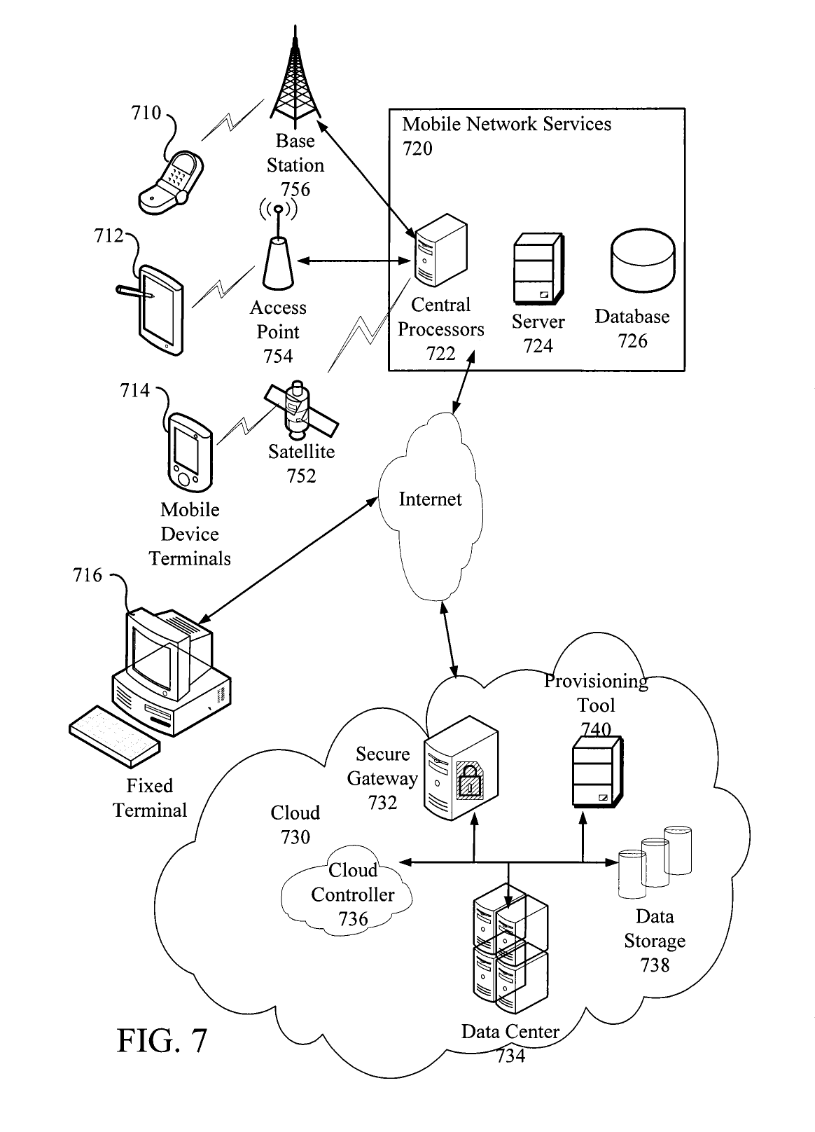

The functions and features described herein may also be executed by various distributed components of a system. For example, one or more processors may execute these system functions, wherein the processors are distributed across multiple components communicating in a network. The distributed components may include one or more client and server machines, which may share processing, as shown on FIG. 7, in addition to various human interface and communication devices (e.g., display monitors, smart phones, tablets, personal digital assistants (PDAs)). The network may be a private network, such as a LAN or WAN, or may be a public network, such as the Internet. Input to the system may be received via direct user input and received remotely either in real-time or as a batch process. Additionally, some implementations may be performed on modules or hardware not identical to those described.

For example, a user, such as medical personnel, may utilize a fixed terminal 716, mobile device 714, PDA 712 and/or phone 710 so as to perform the methods previously described with respect to FIGS. 1-3. Patient information may be stored locally within the memory of a device, or the patient information may be stored remotely over the internet, via a wireline or wireless network connection (via any of satellite 752, access point 754, base station 756, mobile network services 720), at locations such as sever 724 or database 726 of mobile network services 720, data center 734 or data storage 738 of cloud 730.

Processing for the calculation, determination and assessments of the methods described with respect to FIGS. 1-3 may be performed locally by circuitry within a respective device, or may be performed remotely and accessed by a central process 722 or data center 734. Results of processing performed remotely may be transmitted via the internet back to a local device for any output or allocation of hospital resources.

The above-described hardware description is a non-limiting example of corresponding structure for performing the functionality described herein.

Discussion will now turn to experimental results and data analysis performed by the inventors that is significant to the present disclosure.

Preliminary Study

The Functional Status Scale (FSS) was developed by the Collaborative Pediatric Critical Care Research Network funded by the Eunice Kennedy Shriver National Institute of Child Health and Human Development, National Institutes of Health, Department of Health and Human Services: It was based on the Functional Disability Scale (FDS) was initially developed by Dr. Pollack and two developmental psychologists including Dr. Penny Glass. The FSS is utilized for the rapid and reliable assessment of functional status. There are 6 domains including mental status, communication, sensory, motor functioning, feeding, and respiratory status. Each is graded from 1 (normal) to 5 (very severe dysfunction).

TABLE 4
The Functional Status Scale Score

| | | | 3 | 4 | 5 |
|---|---|---|---|---|---|
| | 1 | 2 | MODERATE | SEVERE | VERY SEVERE |
| | NORMAL | MILD DYSFUNCTION | DYSFUNCTION | DYSFUNCTION | DYSFUNCTION |
| MENTAL STATUS | Normal sleep/wake; | Sleepy but arousable to | Lethargic and/or | Minimal arousal to | Unresponsive and/or |
| | appropriate responsivity | noise/touch/movement | irritable | stimulus (stupor) | Coma and/or |
| | | and/or periods of social | | | Vegetative |
| | | nonresponsivity | | | |
| SENSORY | Intact hearing and vision | Suspected hearing or | Not reactive to | Not reactive to | Abnormal response to |
| | and responsive to touch | Suspected vision loss. | auditory stimuli or | auditory stimuli | pain or touch |
| | | | Not reactive to | and Not reactive | |
| | | | visional stimuli | to visional stimuli | |
| COMMUNICATION | Appropriate non-crying | Diminished Vocalization | Absence of attention | No demonstration | Absence of |
| | vocalizations, interactive | Diminished Facial | getting behavior | of discomfort | communication |
| | facial expressiveness, or | Expression and/or social | | | |
| | gestures | responsiveness | | | |
| MOTOR FUNCTION | Coordinated body | 1 limb functionally | 2 or more limbs | Poor head control | Diffuse Spasticity, |
| | movements and normal | impaired | functionally impaired | | Paralysis, |
| | muscle control and | | | | Decerebrate/Decorticate |
| | awareness of action and | | | | Posturing |
| | why it's being done | | | | |
| FEEDING | All food taken by mouth | NPO or need for age- | Oral and tube | Parenteral | All parenteral nutrition |
| | with age appropriate | inappropriate help with | feedings | Nutrition with oral | |
| | help | feeding | | or tube feedings | |
| RESPIRATORY | Room air and no | Oxygen and/or | Tracheostomy | CPAP for all or | Mechanical |
| | artificial support or aids | Suctioning | | part of the day | ventilatory support for |
| | | | | and/or Mechanical | all of the day and |
| | | | | ventilator support | night |
| | | | | for part of the day | |

The FSS was validated on a total of 836 children. Each FSS domain was associated with mean ABAS II (p<0.0001). Discrimination was very good for moderate and severe dysfunction categories (area under the ROC curve >0.8). Intra-class correlations of original FSS was 0.95. Inter-rater reliability was very good to excellent for the FSS domains.

| | Weighted Kappa |
|---|---|
| Mental Status | 0.54 |
| Sensory | 0.76 |
| Communication | 0.81 |
| Motor Functioning | 0.78 |
| Feeding | 0.87 |
| Respiratory | 0.88 |

Studies prior the current studies demonstrated the feasibility of polychotomous outcome prediction in PICU patients.

The inventor of this application has published the following publication, which is hereby incorporated by reference in its entirety: “Simultaneous Prediction of New Morbidity, Mortality, and Survival Without New Morbidity From Pediatric Intensive Care: A New Paradigm for Outcomes Assessment.” Critical Care Medicine; August 2015; Volume 43; Issue 8; pages 1699-1709.

Obviously, numerous modifications and variations of the present disclosure are possible in light of the above teachings. It is therefore to be understood that within the scope of the appended claims, the disclosure may be practiced otherwise than as specifically described herein.

1. A method for determining a patient risk, the method comprising:
obtaining patient information for a patient admitted to a medical facility, the patient information including patient descriptive information, diagnostic information and physiological information;
determining a patient score according to the physiological information; and
simultaneously determining, by circuitry, a patient morbidity risk and a patient mortality risk for the patient according the patient descriptive information, the diagnostic information and the patient score.

2. The method according to claim 1, wherein the determining of the patient score according to the physiological information further comprises:
identifying a numerical value for the patient descriptive information, the diagnostic information and the physiological information; and
determining the patient score by computing the numerical values.

3. The method according to claim 1, wherein the physiological information includes information about a condition of the patient prior to admission to the medical facility.

4. The method according to claim 1, wherein the physiological information includes information about a condition of the patient upon admission to the medical facility.

5. The method according to claim 1, wherein the patient descriptive information includes patient history information.

6. The method according to claim 1, wherein the patient is admitted to an intensive care unit of the medical facility when the patient has a congenital or acquired heart defect, the patient is less than three months of age, and the patient score is determined within a period of 2 hours prior to admission and 4 hours after a cardiac interventional procedure.

7. The method according to claim 1, where the information consisting of the new morbidity and mortality risks of the patient is used by the medical staff/parents/family to better understand the patient's prognosis and to aid in medical decision making.

8. A method for evaluating a quality of care in a hospital, the method comprising:
collecting patient information of a group of patients treated in the hospital over a period of time, the patient information for each patient of the group of patients including patient descriptive information, diagnostic information, and physiological information;
determining, by circuitry, an expected number of patients with new morbidities of the group of patients according to the patient information;
determining an actual number of patients with new morbidities of the group of patients according to the patient information;
determining, by the circuitry, an expected number of mortalities of the group of patients according to the patient information;
determining an actual number of mortalities of the group of patients according to the patient information; and
determining, by the circuitry, the quality of care in the hospital according to the expected and actual number of patients with new morbidities and the expected and actual number of mortalities.

9. The method according to claim 8, wherein the physiological information includes information about a condition of the patient prior to admission to the medical facility.

10. The method according to claim 8, wherein the determining of the expected number of patients with new morbidities comprises:
determining, for each patient of the group of patients, a patient score according to the physiological information;
for each patient, simultaneously determining, by the circuitry, a patient morbidity risk and a patient mortality risk of the patient according the patient descriptive information, the diagnostic information and the patient score; and
calculating, by the circuitry, the expected number of patients with new morbidities according to the patient morbidity risk of each patient in the group of patients.

11. The method according to claim 8, wherein the determining of the patient score according to the physiological information comprises:
identifying a numerical value for the patient descriptive information, the diagnostic information and the physiological information; and
determining the patient score by computing the numerical values.

12. The method according to claim 8, wherein the physiological information includes information about a condition of the patient upon admission to the medical facility.

13. The method according to claim 8, wherein the patient descriptive information includes patient history information.

14. A non-transitory computer readable medium storing computer executable instructions that, when executed by a computer, cause the computer to execute the method according to claim 1.

15. An apparatus, comprising:
circuitry configured to
obtain patient information for a patient, the patient information including patient descriptive information, diagnostic information, and physiological information;
determine a patient score according to the physiological information; and
simultaneously determine a patient morbidity risk and a patient mortality risk for the patient according the patient descriptive information, the diagnostic information and the patient score.

16. The apparatus according to claim 15, wherein to determine the patient score, the circuitry is further configured to
identify a numerical value for the patient descriptive information, the diagnostic information and the physiological information; and
determine the patient score by computing the numerical values.

17. The apparatus according to claim 15, wherein the physiological information includes information about a condition of the patient prior to admission to the medical facility.

18. The apparatus according to claim 15, wherein the circuitry is further configured to
collect patient information for a group of patients over a period of time, the group of patients including the patient;
determine an expected number of patients with new morbidities of the group of patients according to the descriptive information, the diagnostic information, and the physiological information;
determine an actual number of patients with new morbidities of the group of patients according to the patient descriptive information, the diagnostic information and the physiological information;
determine an expected number of mortalities of the group of patients according to the descriptive information, the diagnostic information, and the physiological information;
determine an actual number of mortalities of the group of patients according to the patient descriptive information, the diagnostic information and the physiological information; and
determine the quality of care in the hospital according to the expected and actual number of patients with new morbidities and the expected and actual number of mortalities.

19. The apparatus according to claim 18, wherein to determine the expected number of patients with new morbidities, the circuitry is further configured to
determine, for each patient of the group of patients, a patient score according to the physiological information;
simultaneously determine, for each patient, a patient morbidity risk and a patient mortality risk of the patient according the descriptive information, the diagnostic information, and the patient score; and
calculate the expected number of patients with new morbidities according to the patient morbidity risk and the patient mortality risk of each patient in the group of patients.

20. The apparatus according to claim 15, wherein when the patient is not admitted to an intensive care unit of the medical facility and the patient is over 3 months of age, the circuitry calculates the patient score within a period of 2 hours prior to admission and 4 hours after admission to the medical facility.
